(19) United States
(12) Patent Application Publication
(10) Pub. No.: US 2014/0358040 A1
(43) Pub. Date: Dec. 4, 2014

(54) GAIT MONITORING APPARATUS AND METHOD

(71) Applicant: Electronics And Telecommunications Research Institute, Daejeon (KR)

(72) Inventors: Min-Ho KIM, Daejeon (KR); Ho-Youl JUNG, Daejeon (KR); Myung-Eun LIM, Daejeon (KR); Jae-Hun CHOI, Daejeon (KR)

(73) Assignee: ELECTRONICS AND TELECOMMUNICATIONS RESEARCH INSTITUTE, Daejeon (KR)

(21) Appl. No.: 14/269,029

(22) Filed: May 2, 2014

(30) Foreign Application Priority Data
Jun. 4, 2013 (KR) 10-2013-0063839

(51) Int. Cl. A61B 5/11; A61B 5/00

(52) U.S. Cl. CPC A61B 5/112; A61B 5/6807; A61B 5/7282; A61B 5/742
USPC 600/595

(57) ABSTRACT

Disclosed herein is a gait monitoring apparatus and method. The gate monitoring apparatus includes a preprocessing unit for receiving data from a gait detection sensor and preprocessing the data. A swing detection unit detects, based on the data output from the preprocessing unit, whether a current gait phase is a swing phase in which a foot of a pedestrian is lifted from ground and swings in air when the foot moves forwards. A stance detection unit detects, based on the output data, whether a current gait phase is a stance phase in which the foot is in contact with the ground. A heel-strike detection unit detects, based on the output data, whether a current gait phase is a heel-strike phase in which the heel of the pedestrian strikes ground. A control unit determines the current gait phase, analyzes the gait of the pedestrian, and outputs gait analysis information.

### CROSS REFERENCE TO RELATED APPLICATION

This application claims the benefit of Korean Patent Application No. 10-2013-0063839 filed on Jun. 4, 2013, which is hereby incorporated by reference in its entirety into this application.

### BACKGROUND OF THE INVENTION

1. Technical Field

The present invention relates generally to a gait monitoring apparatus and method and, more particularly, to an apparatus and method that are capable of analyzing and monitoring the gait of a pedestrian in real time.

2. Description of the Related Art

The necessity of devices for measuring and analyzing the motion of a part of a body and devices for applying such a device has come to the fore in the sports and health care industries.

In particular, the movement of legs, that is, a gait, is associated with a proprioceptive sensation that may be regarded as an inner sensation, a motor nervous system, and a vertebrate nervous system, as well as the five senses. Therefore, if an abnormality occurs in any one of these systems, a gait pattern differing from that of a normal person is exhibited.

In the area of kinematic analysis, kinematic data (the relative angle of joints) or dynamic data (the force of joints, moment) produced from joints has become criteria for determining whether an abnormality is present via the analysis of human movement.

Prior art related to the analysis of a gait is disclosed in Korean Patent Application Publication No. 2013-0029223 (entitled “Gait training system, a data processing apparatus thereof, and a method of operating the data processing apparatus”) which senses whether a parallel-footed gait has occurred, and provides information about the occurrence of the parallel-footed gait to a pedestrian.

The invention disclosed in Korean Patent Application Publication No. 2013-0029223 includes a wireless communication unit for receiving acceleration data from a gait sensor device, a parallel-footed gait detection unit for determining using the acceleration data whether the pedestrian walks in a parallel-footed gait pattern, and a display unit for providing information about the determination of the parallel-footed gait pattern to the pedestrian.

When the gait training system described in Korean Patent Application Publication No. 2013-0029223 is actually implemented, a normal person must be able to get gait training anywhere and at anytime without environmental restrictions such as having to be within a specific indoor or outdoor area, or temporal restrictions such as having to be at a specific time. That is, such a system must be implemented as a compact device which is as small as possible so that a user can easily put on the device without inconvenience, and must be equipment which can provide a long operational duration to such an extent that the user cannot feel inconvenience caused by the charging of the equipment.

One of essential technologies for satisfying such preconditions is technology for analyzing a gait at low power and in real time. The above invention described in Korean Patent Application Publication No. 2013-0029223 describes only whether a parallel-footed gait occurs, and does not describe technology for analyzing a gait at low power and in real time.

Further, conventional gait analysis technologies require a very-high sampling rate (the number of samples per second) (several hundred Hz), but these characteristics become an obstacle to low power implementation. That is, for low power implementation, there is required technology that is capable of analyzing a gait using data sampled at a relatively low sampling rate (the number of samples per second) which enables sampling.

### SUMMARY OF THE INVENTION

Accordingly, the present invention has been made keeping in mind the above problems occurring in the prior art, and an object of the present invention is to provide a gait monitoring apparatus and method, which can process gait analysis at low power and in real time using data sampled at a relatively low sampling rate (the number of samples per second) upon analyzing a gait using a 3-axis acceleration sensor.

In accordance with an aspect of the present invention to accomplish the above object, there is provided a gait monitoring apparatus, including a preprocessing unit for receiving data from a gait detection sensor and preprocessing the data; a swing detection unit for detecting, based on the data output from the preprocessing unit, whether a current gait phase is a swing phase in which a foot of a pedestrian is lifted from ground and swings in air when the foot of the pedestrian moves forwards; a stance detection unit for detecting, based on the data output from the preprocessing unit, whether a current gait phase is a stance phase in which the foot of the pedestrian is in contact with the ground; a heel-strike detection unit for detecting, based on the data output from the preprocessing unit, whether a current gait phase is a heel-strike phase in which the heel of the pedestrian strikes the ground; and a control unit for determining the current gait phase of the pedestrian, analyzing the gait of the pedestrian, and outputting gait analysis information, based on information output from the preprocessing unit, the swing detection unit, the stance detection unit, and the heel-strike detection unit.

Preferably, the preprocessing unit may receive sampling data at a sampling rate of 20 to 50 Hz from the gait detection sensor.

Preferably, the preprocessing unit may receive acceleration data on X, Y, and Z axes from the gait detection sensor, and calculate values of a plurality of variables based on the acceleration data.

Preferably, the plurality of variables may include a variable indicative of energy on XZ axes, a variable indicative of energy on a Y axis, a variable indicative of a product of acceleration data in directions of the X axis and Z axis, and a variable indicative of a variation in the direction of the Z axis.

Preferably, the preprocessing unit may set a value, generated by summing up a square of an offset-calibrated value in the direction of the X axis and a square of an offset-calibrated value in the direction of the Z axis, to a value of the variable indicative of energy on the XZ axes.

Preferably, the preprocessing unit may set a value, generated by squaring an offset-calibrated value in the direction of the Y axis, to a value of the variable indicative of energy on the Y axis.

Preferably, the preprocessing unit may set a value, generated by multiplying an offset-calibrated value in the direction of the X axis by an offset-calibrated value in the direction of the Z axis, to a value of the variable indicative of the product of the acceleration data in the directions of the X and Z axes.

Preferably, the swing detection unit may be configured to, if a value of the variable indicative of energy on the XZ axes is equal to or greater than a preset minimum energy threshold in a non-stance phase, and if a value of the variable indicative of the product of the acceleration data in the directions of the X and Z axes is a negative value, detect the current gait phase of the pedestrian as the swing phase.

Preferably, the control unit may determine, based on swing phase detection information output from the swing detection unit, that the current gait phase of the pedestrian is the swing phase, and that a previous gait event is a toe off event.

Preferably, the stance detection unit may be configured to, if a value of the variable indicative of energy on the XZ axes is less than a preset minimum energy threshold in a non-stance phase, and a count value for a low energy state is equal to or greater than a preset minimum threshold for the low energy count, detect the current gait phase of the pedestrian as the stance phase.

Preferably, the control unit may determine, based on stance phase detection information output from the stance detection unit, that the current gait phase of the pedestrian is the stance phase, and that a previous gait event is a toe ground event.

Preferably, the heel-strike detection unit may be configured such that, after it is determined that a value of the variable indicative of energy on the XZ axes is greater than a preset minimum threshold for heel-strike energy and that a value of the variable indicative of the product of the acceleration data in the directions of the X and Z axes is greater than a value generated by dividing the value of the variable indicative of energy on the XZ axes by a predetermined value, if the value of the variable indicative of energy on the XZ axes is less than a value generated by dividing a previous value of the variable indicative of energy on the XZ axes by a predetermined value, a previous gait event of the pedestrian is detected as the heel-strike phase.

Preferably, the apparatus may further include an offset recalculation unit for recalculating an offset for the data output from the preprocessing unit.

Preferably, the offset recalculation unit may determine whether a current state is a state potentially requiring offset recalculation, based on one of a variable indicative of a variation in the direction of the X axis, a variable indicative of a variation in the direction of the Y axis, or a variable indicative of a variation in the direction of the Z axis, and perform offset recalculation if the state potentially requiring offset recalculation is maintained for a predetermined period of time or longer, wherein the variables indicative of the variations are output from the preprocessing unit.

Preferably, the control unit may analyze whether the pedestrian walks in a parallel-footed gait pattern, based on values of the variable indicative of energy on the XZ axes and the variable indicative of energy on the Y axis, and outputs gait analysis information to a display unit.

In accordance with another aspect of the present invention to accomplish the above object, there is provided a gait monitoring method, including receiving, by a preprocessing unit, data from a gait detection sensor and preprocessing the data; detecting, by a swing detection unit, whether a current gait phase is a swing phase in which a foot of a pedestrian is lifted from ground and swings in air when the foot of the pedestrian moves forwards, based on the data at preprocessing; detecting, by a stance detection unit, whether a current gait phase is a stance phase in which the foot of the pedestrian is in contact with the ground, based on the data at preprocessing; detecting, by a heel-strike detection unit, whether a current gait phase is a heel-strike phase in which the heel of the pedestrian strikes the ground, based on the data at preprocessing; and determining, by a control unit, the current gait phase of the pedestrian, analyzing the gait of the pedestrian, and outputting gait analysis information, based on information obtained at preprocessing, at detecting the swing phase, at detecting the stance phase, and at detecting the heel-strike phase.

Preferably, detecting whether the current gait phase is the swing phase may be configured to, if a value of the variable indicative of energy on the XZ axes, generated at preprocessing, is equal to or greater than a preset minimum energy threshold in a non-stance phase, and if a value of the variable indicative of the product of the acceleration data in the directions of the X and Z axes, generated at preprocessing, is a negative value, detect the current gait phase of the pedestrian as the swing phase.

Preferably, detecting whether the current gait phase is the stance phase may be configured to, if a value of the variable indicative of energy on the XZ axes, generated at preprocessing, is less than a preset minimum energy threshold in a non-stance phase, and a count value for a low energy state is equal to or greater than a preset minimum threshold for the low energy count, detect the current gait phase of the pedestrian as the stance phase.

Preferably, detecting whether the current gait phase is the heel-strike phase may be configured such that, after it is determined that a value of the variable indicative of energy on the XZ axes, generated at preprocessing, is greater than a preset minimum threshold for heel-strike energy and that a value of the variable indicative of the product of the acceleration data in the directions of the X and Z axes, generated at preprocessing, is greater than a value generated by dividing the value of the variable indicative of energy on the XZ axes by a predetermined value, if the value of the variable indicative of energy on the XZ axes is less than a value generated by dividing a previous value of the variable indicative of energy on the XZ axes by a predetermined value, a previous gait event of the pedestrian is detected as the heel-strike phase.

Preferably, outputting the gait analysis information may be configured to analyze whether the pedestrian walks in a parallel-footed gait pattern, based on values of the variable indicative of energy on the XZ axes and the variable indicative of energy on the Y axis, the variables being generated at preprocessing.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing a gait cycle applied to an embodiment of the present invention;

FIG. 2 is a block diagram showing a gait monitoring apparatus according to an embodiment of the present invention;

FIG. 3 is a flowchart schematically showing a gait monitoring method according to an embodiment of the present invention;

FIG. 4 is a flowchart showing in detail the preprocessing task procedure of FIG. 3;

FIG. 5 is a flowchart showing in detail the swing detection procedure of FIG. 3;

FIGS. 6A and 6B are flowcharts showing in detail the stance detection procedure of FIG. 3;

FIG. 7 is a flowchart showing in detail a procedure for calculating the sum of energies in the directions of X and Z axes and calculating the sum of energies in the direction of a Y axis by using values stored in a buffer in FIG. 6A;

FIG. 8 is a flowchart showing in detail the offset recalculation procedure of FIG. 3; and

FIGS. 9A and 9B are flowcharts showing in detail the heel-strike detection procedure of FIG. 3.

FIG. 10 is an embodiment of the present invention implemented in a computer system.

### DESCRIPTION OF THE PREFERRED EMBODIMENTS

A gait monitoring apparatus and method according to embodiments of the present invention will be described below with reference to the accompanying drawings. Prior to the following detailed description of the present invention, it should be noted that the terms and words used in the specification and the claims should not be construed as being limited to ordinary meanings or dictionary definitions. Meanwhile, the embodiments described in the specification and the configurations illustrated in the drawings are merely examples and do not exhaustively present the technical spirit of the present invention. Accordingly, it should be appreciated that there may be various equivalents and modifications that can replace the embodiments and the configurations at the time at which the present application is filed.

Below, a gait cycle to be applied to an embodiment of the present invention will be described.

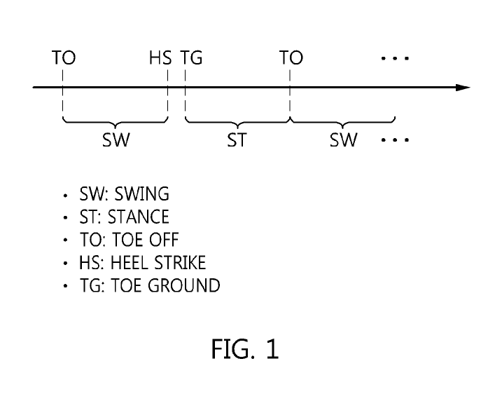

In order to describe gait analysis, a gait cycle must be first understood. FIG. 1 illustrates gait phases and gait events handled in the embodiment of the present invention. In detail, gait phases may include a swing (SW) phase indicating a phase in which the foot of a pedestrian is lifted from the ground and swings in the air when the foot moves forwards, and a stance phase (ST) in which the entire foot is in contact with the ground. Further, gait events may include a toe off (TO) event, a heel-strike (HS) event, and a toe ground (TG) event.

In other words, gait events are circulated in the sequence of “toe off (TO)→heel-strike (HS)→toe ground (TG)→toe off (TO)→ . . . .” In this case, a period corresponding to “toe off (TO)→heel-strike (HS)” may be defined as a swing (SW) period, and a period corresponding to “toe ground (TG)→toe off (TO)” may be defined as a stance (ST) period.

Further, depending on the circumstances, the swing period may be defined as a period except the stance period, that is, a period corresponding to “toe off (TO)→heel-strike (HS)→toe ground (TG)”.

The stance phase in which the foot of the pedestrian is in contact with the ground may be variously defined. For example, the stance phase may correspond to a time ranging from a point in time at which the toes of the pedestrian touch the ground after the heel of the pedestrian strikes the ground during a gait action to a point in time at which the toes of the pedestrian leave the ground. In another embodiment, the stance phase in which the foot of the pedestrian is in contact with the ground may correspond to a time ranging from a point in time at which the toes of the pedestrian touch the ground during a gait action to a point in time at which the toes leave the ground.

In more detailed gait analysis, a larger number of gait phases and events will be handled. However, since the present invention is intended to perform low-power and real-time gait analysis, the number of gait phases and gait events to be detected is minimized. The sampling rate (the number of samples per second) falls within a range in which sampling is possible at low power, and may increase with the development of low-power sampling technology. In an embodiment of the present invention, 20 to 50 times per second, that is, a frequency of 20 to 50 Hz (preferably, 30 Hz), is assumed and described.

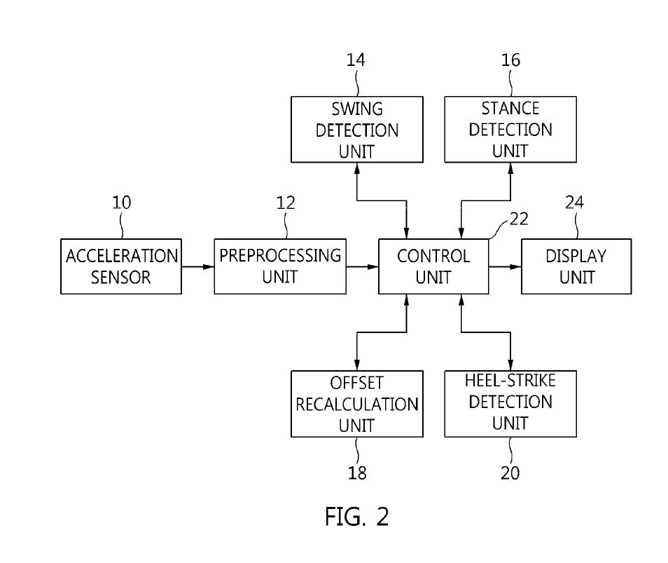

FIG. 2 is a block diagram showing a gait monitoring apparatus according to an embodiment of the present invention.

The gait monitoring apparatus according to the embodiment of the present invention includes an acceleration sensor 10, a preprocessing unit 12, a swing detection unit 14, a stance detection unit 16, an offset recalculation unit 18, a heel stance detection unit 20, a control unit 22, and a display unit 24.

The acceleration sensor 10 is mounted on each of the shoes of the pedestrian, and is preferably installed in a portion corresponding to the top of each foot of the pedestrian or a region under the metatarsals of the foot when the pedestrian puts on his or her shoes. The acceleration sensor 10 measures accelerations on 3 axes upon walking, and provides the measured accelerations to the preprocessing unit 12. That is, the acceleration sensor 10 measures acceleration values on 3 axes when the pedestrian walks. Here, the three axes denote an X axis, a Y axis, and a Z axis. For example, the X axis denotes the movement direction of the pedestrian, the Y axis denotes a horizontal direction with respect to the movement direction of the pedestrian, and the Z axis denotes a vertical direction with respect to the movement direction of the pedestrian. The acceleration sensor 10 transmits the measured acceleration values on the 3 axes to the preprocessing unit 12 in a wireless manner. In other words, the acceleration sensor 10 may convert the measured acceleration values on the 3 axes into digital values and transmit the digital acceleration values to the preprocessing unit 12 in a wireless manner. The acceleration sensor 10 may be an example of a gait detection sensor described in the accompanying claims of the present invention.

The preprocessing unit 12 receives sampling data at a sampling rate of 20 to 50 Hz (preferably, 30 Hz) from the acceleration sensor 10, and preprocesses the sampling data. Here, the term “preprocessing” denotes the procedure of generating the values of a variety of variables required for analysis before the data from the acceleration sensor 10 is analyzed. The preprocessing unit 12 receives acceleration data on the X, Y, and Z axes from the acceleration sensor 10, and calculates the values of a plurality of variables (also referred to as ‘parameters’) based on the acceleration data on the X, Y, and Z axes. Here, the plurality of variables may include a variable energyXZ indicative of energy (kinetic energy) on the X axis and the Z axis (hereinafter referred to as ‘XZ axes’), a variable energyY indicative of energy (kinetic energy) on the Y axis, a variable (product) indicative of a multiplication (product) of acceleration data values on the X and Z axes, and a variable gapZ indicative of a variation from a previous value on the Z axis. Of course, the plurality of variables may additionally include a variable gapX indicative of a variation from a previous value on the X axis, and a variable (gapY) indicative of a variation from a previous value on the Y axis.

In more detail, the preprocessing unit 12 may set a value (a), generated by summing up the square of an offset-calibrated value (x−oX) in the direction of the X axis and the square of an offset-calibrated value (z−oZ) in the direction of the Z axis, to the value (a) of the variable (energyXZ) indicative of energy on the XZ axes. If this procedure is represented by an equation, “energyXZ=(x−oX)*(x−oX)+(z−oZ)*(z−oZ)” may be obtained.

Meanwhile, the preprocessing unit 12 may set a value (b), generated by squaring an offset-calibrated value (y−oY) in the direction of the Y axis, to the value (b) of the variable energyY indicative of energy on the Y axis. If this is represented by an equation, “energyY=(y−oY)*(y−oY)” may be obtained.

Further, the preprocessing unit 12 may set a value (c), generated by multiplying the offset-calibrated value (x−oX) in the direction of the X axis by the offset-calibrated value (z−oZ) in the direction of the Z axis, to the value (c) of the variable (product) indicative of the product of acceleration data in the directions of the X and Z axes. If this procedure is represented by an equation, “product=(x−oX)*(z−oZ)” may be obtained.

Meanwhile, the preprocessing unit 12 may set, for example, a difference (d) between a current input data value z and a previous value prevZ to the value (d) of the variable gapZ indicative of the variation from the previous value on the Z axis. If this procedure is represented by an equation, “gapZ=z−prevZ” may be obtained.

The above-described preprocessing unit 12 may occasionally perform zero-point adjustment (calibration) on the acceleration data on the three axes. In order to perform calibration, offset values required to respectively correct acceleration values on the X, Y, and Z axes are required. For example, the preprocessing unit 12 may set the offset values oX, oY, and oZ of the X, Y, and Z axes to acceleration values measured when the foot of the pedestrian is in contact with the ground.

The swing detection unit 14 determines, based on the data output from the preprocessing unit 12, whether the foot of the pedestrian is in the swing (SW) phase in which the foot is lifted from the ground and swings in the air when moving forwards. In other words, the swing detection unit 14 may determine that the current gait phase of the pedestrian is the swing phase if the value of the variable energyXZ indicative of energy on the XZ axes, among the pieces of data from the preprocessing unit 12, is equal to or greater than a preset minimum energy threshold MIN_NO_ST_ENERGY in a non-stance phase, and if the value of the variable (product) indicative of the product of acceleration data in the directions of the X axis and Z axis is a negative value, thus detecting the swing phase. Further, the swing detection unit 14 transmits swing phase detection information to the control unit 22. Accordingly, the control unit 22 may determine, based on the swing phase detection information from the swing detection unit 14, that the current gait phase of the pedestrian is the swing phase, and may determine (recognize) that a previous gait event is a toe off (TO) event. The above-described minimum energy threshold MIN_NO_ST_ENERGY in the non-stance phase may be a maximum allowable variation in energy values in the stance (ST) phase and may be set to, for example, “0x20000.”

The stance detection unit 16 detects, based on the data output from the preprocessing unit 12, whether a current gait phase is a stance phase in which the foot of the pedestrian is in contact with the ground. In other words, the stance detection unit 16 recognizes that a current energy state is a low energy state if the variable energyXZ indicative of energy on the XZ axes, among pieces of data from the preprocessing unit 12, is less than the preset minimum energy threshold MIN_NO_ST_ENERGY in the non-stance phase. The stance detection unit 16 is configured to, if a count value LECount for the low energy state is equal to or greater than a preset low energy count minimum threshold MIN_LE_COUNT, determine that the current gait phase of the pedestrian is the stance (ST) phase, thus detecting the stance phase. Further, the stance detection unit 16 transmits stance phase detection information to the control unit 22. Accordingly, the control unit 22 may determine, based on the stance phase detection information from the stance detection unit 16, that the current gait phase of the pedestrian is the stance (ST) phase, and may determine (recognize) that a previous gait event is a toe ground (TG) event. The preset low energy count minimum threshold MIN_LE_COUNT may be, for example, “7.” That is, if the value of the variable energyXZ indicative of energy on the XZ axes is less than the preset minimum energy threshold MIN_NO_ST_ENERGY in the non-stance phase, the current energy state is recognized as a low energy state, and such a low energy state is counted. For example, a state in which the value of the variable energyXZ is equal to or less than the exemplified preset minimum energy threshold MIN_NO_ST_ENERGY in the non-stance phase may be regarded as the low energy state. Therefore, the stance detection unit 16 may count such a low energy state, and may set a value obtained by counting the low energy state to the value of the variable LECount.

The offset recalculation unit 18 recalculates the offset of data output from the preprocessing unit 12. The offset recalculation unit 18 determines whether a current state is a state (QST) potentially requiring offset recalculation, by using one selected from among the variable gapX indicative of a variation from a previous value on the X axis, the variable gapY indicative of a variation from a previous value on the Y axis, and the variable gapZ indicative of a variation from a previous value on the Z axis. If the state QST potentially requiring offset recalculation is maintained for a predetermined period of time or longer, the offset is recalculated.

The heel-strike detection unit 20 detects, based on the data output from the preprocessing unit 12, whether the current phase is a heel-strike phase in which the heel of the pedestrian strikes the ground. After it is determined that the value of the variable energyXZ indicative of energy on the XZ axes is greater than a preset heel-strike energy minimum threshold MIN_HS_ENERGY, and that the value of the variable (product) indicative of the product of the acceleration data in the directions of the X and Z axes is greater than the value obtained by dividing the value of the variable energyXZ indicative of energy on the XZ axes by a predetermined value, if it is determined that the value of the variable energyXZ indicative of energy on the XZ axes is less than a value obtained by dividing the previous value prevEnergyXZ of the variable energyXZ indicative of energy on the XZ axes by a predetermined value, the heel-strike detection unit 20 detects that the previous gait event of the pedestrian is the heel-strike phase.

The term “detection” in the description of the swing detection unit 14, the stance detection unit 16, and the heel-strike detection unit 20 may be replaced with the term “determination.” Further, the detection of a swing period may be understood to be the detection of a swing phase, and the detection of a stance period may be understood to be the detection of a stance phase.

The control unit 22 transmits the data of the preprocessing unit 12 to the swing detection unit 14, the stance detection unit 16, the offset recalculation unit 18, and the heel-strike detection unit 20. The control unit 22 determines the current gait phase of the pedestrian, analyzes the gait of the pedestrian, and outputs gait analysis information, based on the information output from the preprocessing unit 12, the swing detection unit 14, the stance detection unit 16, and the heel-strike detection unit 20.

For example, the control unit 22 analyzes whether the pedestrian walks in a parallel-footed gait pattern, based on the values of the variable energyXZ indicative of energy on the XZ axes and the variable energyY indicative of energy on the Y axis, and then outputs gait analysis information to the display unit 24. That is, the control unit 22 may analyze whether the pedestrian walks in the parallel-footed gait pattern, using the sum of the values of the variable energyXZ indicative of energy on the XZ axes and the sum of the values of the variable energyY indicative of energy on the Y axis. For example, if the pedestrian walks in a non-parallel-footed gait pattern (for example, an out-toed gait), a motion in a horizontal direction (y axis direction) with respect to the movement direction (x axis direction) becomes relatively large. Therefore, the control unit 22 may determine whether the pedestrian walks in the parallel-footed gait pattern by detecting the relative intensity of the horizontal motion while walking, based on the acceleration data on the three axes. For example, the control unit 22 may determine whether the pedestrian walks in the parallel-footed gait pattern, using the equation “K=energySumXZ/energySumY.” Here, energySumXZ may be understood to be the sum of the values of the variable energyXZ generated during a swing (SW) period, and energySumY may be understood to be the sum of the values of the variable energyY generated during the swing (SW) period. That is, K is calculated using the acceleration values on the X, Y, and Z axes sampled at respective times during the swing period. For example, if sampling occurs ten times during the swing period, the sums of energy values calculated from the acceleration values on the X, Y, and Z axes for ten times become energySumXZ and energySumY. In accordance with the above equation, K has a smaller value as a gait pattern deviates from a parallel-footed gait, whereas K has a larger value as the gait pattern is closer to the parallel-footed gait. The determination of whether the gait pattern is a parallel-footed gait may be performed by checking the magnitude of K. Alternatively, if the value of K is less than a predefined threshold, it may be determined that the gait pattern is a non-parallel-footed gait, whereas if the value of K is greater than the threshold, it may be determined that the gait pattern is a parallel-footed gait.

Meanwhile, the control unit 22 may update pieces of statistical data related to K. For example, if it is assumed that, whenever the gait of a pedestrian is recognized, the average degree of the parallel-footed gait is displayed to the pedestrian, the control unit 22 may calculate and update statistical data related to K. In this case, the statistical data related to K may be displayed on the display unit 24 in the form of “mean/standard deviation of K for recent several minutes”, “mean/standard deviation of K during current parallel-footed gait training,” and “mean/standard deviation of K during parallel-footed gait training done for one recent week.”

The display unit 24 receives gait analysis information from the control unit 22 and displays the gait analysis information. For example, the display unit 24 may display to the pedestrian, in real time, information about whether his or her gait pattern is a parallel-footed gait. In another example, the display unit 24 may display the degree of the parallel-footed gait to the pedestrian whenever the step of the pedestrian is recognized. In a further example, the display unit 24 may display both the real-time determination of whether the gait pattern is a parallel-footed gait and the degree of the parallel-footed gait to the pedestrian. Further, the display unit 24 may notify the pedestrian whether the gait pattern is a parallel-footed gait, by means of sound, vibration, or the like.

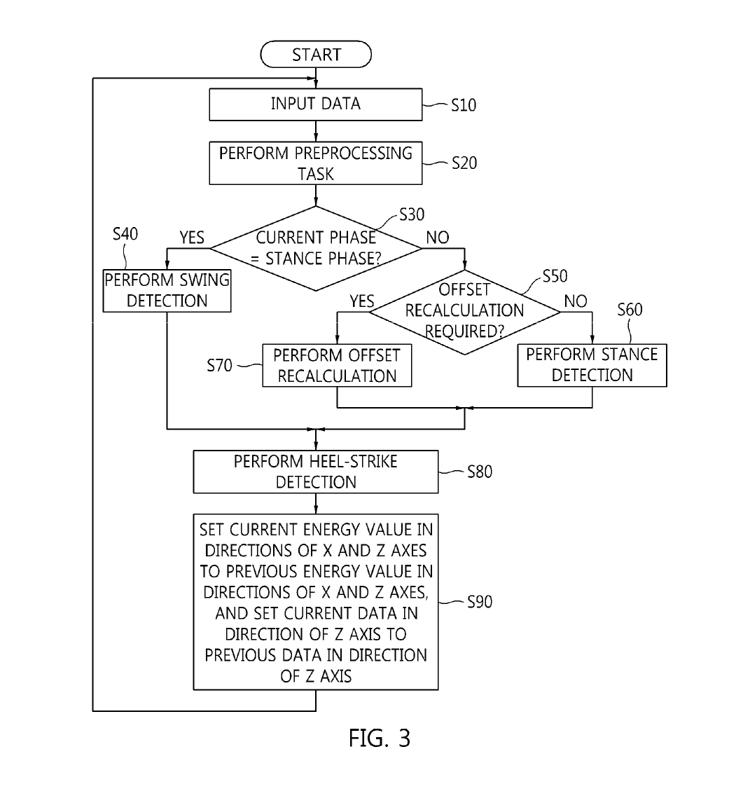

FIG. 3 is a flowchart schematically showing a gait monitoring method according to an embodiment of the present invention.

First, when the gait of a pedestrian is initiated, sampled data (acceleration data on X, Y, and Z axes) from the acceleration sensor 10 is continuously input to the preprocessing unit 12 at step S10.

The preprocessing unit 12 performs the preprocessing task of calculating the values of a plurality of variables based on the input acceleration data on the X, Y, and Z axes at step S20.

The data generated by the preprocessing task of the preprocessing unit 12 is transferred to the control unit 22, and the control unit 22 transmits the input data to the swing detection unit 14, the stance detection unit 16, the offset recalculation unit 18, and the heel-strike detection unit 20, thus enabling swing detection, stance detection, offset recalculation, and heel-strike detection to be performed.

If a current gait phase is a stance (ST) phase (Yes at step S30), a swing (SW) phase is detected after the detection of the stance phase at step S40. That is, the fact that the current gait phase is the stance (ST) phase means that the stance (ST) phase has already been detected, and thus there is no need to again detect the stance (ST) phase, and a swing (SW) phase is detected so as to determine whether a change to the swing (SW) phase has occurred.

In contrast, if the current gait phase is not a stance (ST) phase (No at step S30), the current gait phase will be a swing (SW) phase, and thus it is determined whether a current state is a state requiring offset recalculation at step S50.

If it is determined that the current state is not a state requiring offset recalculation, a stance (ST) phase is detected at step S60, whereas if it is determined that the current state is the state requiring offset recalculation, offset recalculation is performed at step S70.

Meanwhile, since heel-strike (HS) detection is performed subsequent to the swing (SW) phase in a gait cycle, it is natural that heel strike (HS) is detected in the swing (SW) phase, but the HS is detected separately from the swing (SW) phase so as to improve the precision of detection at step S80.

Thereafter, the value of the variable energyXZ generated by the preprocessing unit 12 is set to the value of a variable prevEnergyXZ indicative of previous energy in the directions of the XZ axes, and input data z in the direction of the Z axis is set to previous input data prevZ at step S90.

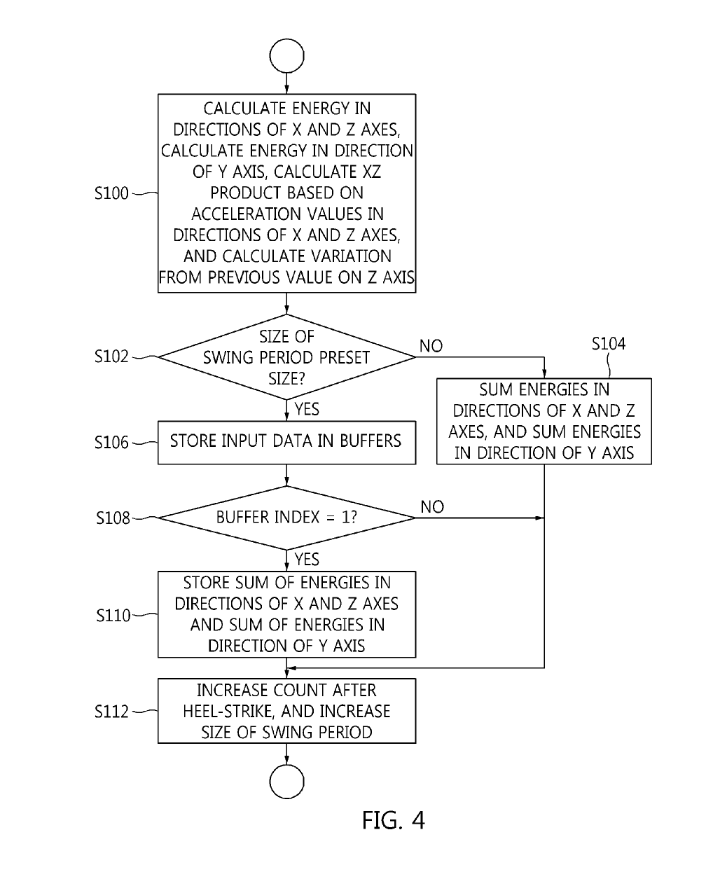

FIG. 4 is a flowchart showing in detail the preprocessing task procedure of FIG. 3.

When pieces of data (x, y, z) are input from the acceleration sensor 10, the preprocessing unit 12 performs various preprocessing tasks.

First, the preprocessing unit 12 calculates energy in the directions of X and Z axes, energy in the direction of the Y axis, an XZ product based on the acceleration values in the directions of the X and Z axes, and a variation from a previous value on the Z axis at step S100. Here, the calculated energy in the directions of the X and Z axes is set to the value of a variable energyXZ, the calculated energy in the direction of the Y axis is set to the value of a variable energyY, the XZ product based on the acceleration values in the directions of the X and Z axes is set to the value of a variable (product), and the variation from the previous value on the Z axis is set to the value of a variable gapZ. In this case, it is noted that the input acceleration data values are not used without change, and are calculated after offsets (oX, oY, oZ) are respectively subtracted from the acceleration data values. Even in the stance phase, since the values of the acceleration sensor 10 do not have a value of “0 (zero)”, offset-calibrated acceleration values are used in the calculation of energies and the XZ product so as to compensate for such offsets.

Then, the preprocessing unit 12 is configured to, if the size of the swing period (wsSW: window size of SWing) is less than a preset size MIN_SW_LENGTH (No at step S102), immediately calculate the values of the variables energySumXZ and energySumY at step S104. Here, the preset size MIN_SW_LENGTH may be preset to about “6.”

In contrast, the preprocessing unit 12 is configured to, if the size of the swing period wsSW is equal to or greater than the preset size MIN_SW_LENGTH (Yes at step S102), store the input data (x, y, z) in buffers bufX[idx], bufY[idx], and bufZ[idx], respectively, without immediately applying the currently input acceleration values energySumXZ and energySumY to calculation, at step S106. Of course, the preprocessing unit 12 stores the input data (x, y, z) in the buffers bufX[idx], bufY[idx], and bufZ[idx], respectively, while sequentially increasing the index idx of each buffer. In this way, if the size of the swing period wsSW is equal to or greater than the preset size MIN_SW_LENGTH, actual calculation using the variables energySumXZ and energySumY is performed after the stance has been detected. The reason for this is that it is difficult to immediately detect a period between the heel-strike (HS) and toe ground (TG), which occur subsequent to the swing (SW) period and in which energy variation equal to or greater than that of the swing (SW) period is caused, and the stance (ST) period. These characteristics are associated with delaying a detection time so as to detect the stance period or the like using a minimum computational load. Therefore, the calculation of energy must be delayed after the stance (ST) period and the toe ground (TG) time have been detected.

Further, the preprocessing unit 12 sets the sum of energies in the swing (SW) period, obtained until data starts to be stored in the buffers bufX[idx], bufY[idx], and bufZ[idx] to base values energySumBaseXZ and energySumBaseY, and allows the base values to be used when the sum of all energies is subsequently calculated at steps S108 and S110. Here, the base value energySumBaseXZ is the base value of the sum of energies in the directions of X and Z axes, and the value of the variable energySumXZ is used as the base value energySumBaseXZ. The base value energySumBaseY is the base value of the sum of energies in the direction of the Y axis, and the value of the variable energySumY is used as the base value energySumBaseY.

It may be understood that a plurality of variables generated in the above preprocessing task procedure may be obtained by the preprocessing unit 12, and the buffers bufX[idx], bufY[idx], and bufZ[idx] are contained in the preprocessing unit 12.

Thereafter, the preprocessing unit 12 increases a count value after heel-strike (AHSCount: After Heel-strike Count), and increases the size of the swing period wsSW at step S112.

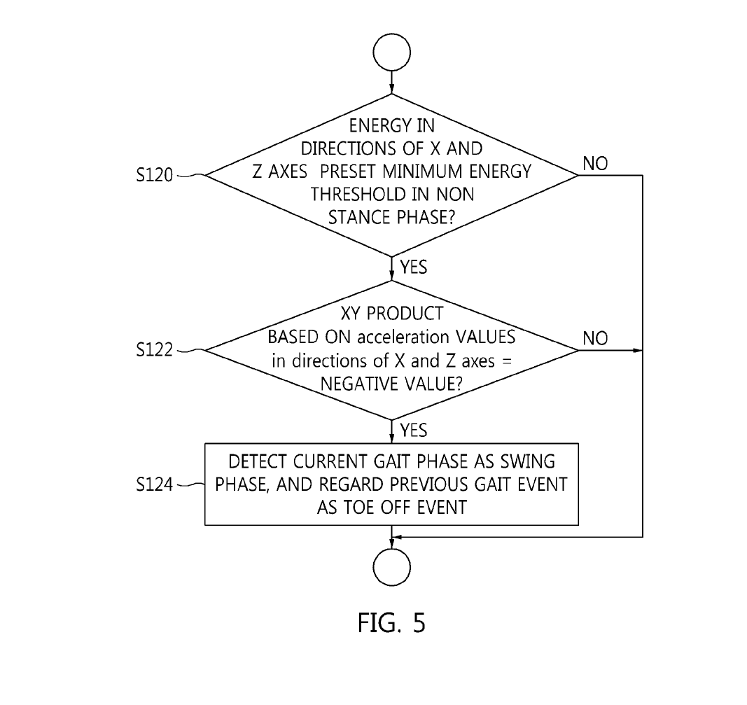

FIG. 5 is a flowchart showing in detail the swing detection procedure of FIG. 3. The swing detection procedure may be regarded as being performed by the swing detection unit 14.

FIG. 5 illustrates a swing detection algorithm and may be regarded as describing, in detail, an algorithm for detecting toe off (TO). The reason for this is that a current gait phase (state) is changed to a swing (SW) phase while detecting toe off (TO).

The swing detection unit 14 detects swing (SW) (that is, the moment of detection of toe off (TO)) in such a way as to detect the current gait phase of the pedestrian as a swing (SW) phase if the value of the variable energyXZ is equal to or greater than a preset minimum energy threshold MIN_NO_ST_ENERGY in a non-stance phase (Yes at step S120), and if the value of the variable (product) indicative of the product of acceleration data in the directions of the X axis and Z axis is a negative value (Yes at step S122), and notifies the control unit 22 of the detection of the swing phase (S124). Accordingly, the swing detection unit 14 and/or the control unit 22 regard a previous gait event as a toe off (TO) event. Together with this, the size of the swing period wsSW is set to “1”, the value of the variable energyXZ is set to the value of the variable energySumXZ, the value of the variable energyY is set to the value of the variable energySumY, the index idx of each buffer is set to “0 (zero)”, and the value of a variable idxHS is set to “0 (zero).”

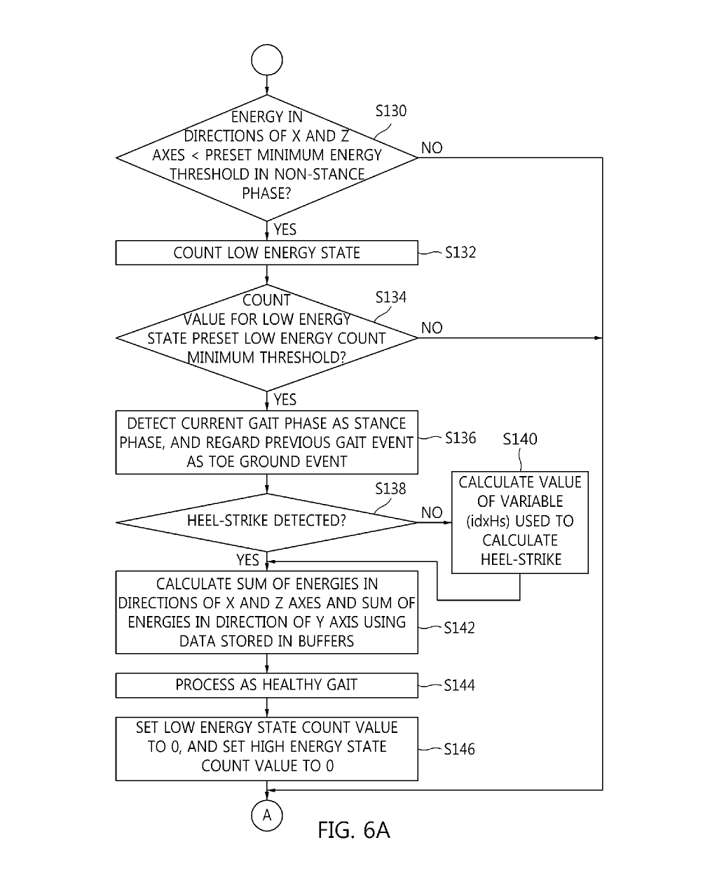

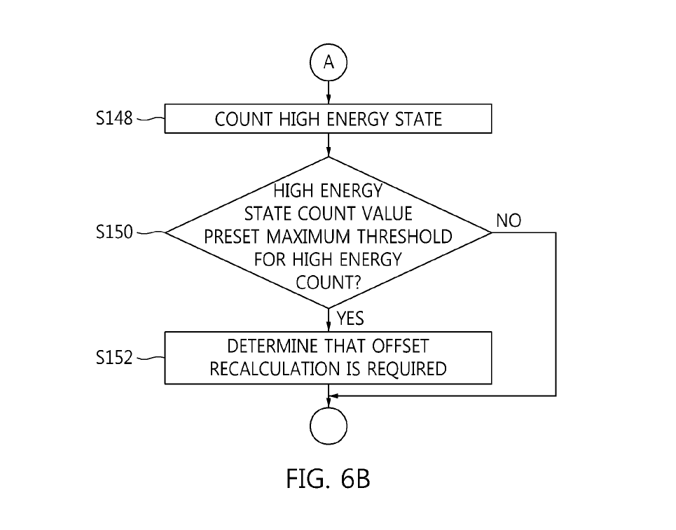

FIGS. 6A and 6B are flowcharts showing in detail the stance detection procedure of FIG. 3. The stance (ST) detection procedure may be regarded as being performed by the stance detection unit 16.

Even in stance (ST) detection, the main object thereof is to perform detection with a minimum computational load, similar to the case of any other detection. For this, only low energy (LE) states are counted, and it is determined whether a count value exceeds a predetermined value MIN_LE_COUNT.

That is, the stance detection unit 16 determines whether the value of the variable energyXZ indicative of energy on the XZ axes is less than a preset minimum energy threshold MIN_NO_ST_ENERGY in a non-stance phase at step S130.

Whenever the variable energyXZ indicative of energy on the XZ axes is less than the preset minimum energy threshold MIN_NO_ST_ENERGY in the non-stance phase, it is determined that a current energy state is a low energy state, and thus a count value for the low energy state LECount is increased by 1 at step S132.

Further, the stance detection unit 16 determines whether the count value for the low energy state LECount is equal to or greater than a preset low energy count minimum threshold MIN_LE_COUNT at step S134.

If it is determined that the count value for the low energy state LECount is equal to or greater than the preset low energy count minimum threshold MIN_LE_COUNT, the stance detection unit 16 detects the current gait phase of the pedestrian as a stance (ST) phase, and notifies the control unit 22 of the detection of the stance phase at step S136. Once the stance (ST) phase is detected, a previous gait event may be regarded as a toe ground (TG) event.

Meanwhile, one thing to be noted is to separate a case where heel-strike (HS) is detected from the remaining cases. The reason for this is to improve fault tolerance by normally performing a processing procedure, which is to be performed after the detection of the stance (ST) phase, even when heel-strike (HS) is not detected.

If heel-strike (HS) has not been detected before stance (ST) phase is detected (No at step S138), a variable (idxHS: index required to calculate heel-strike) is calculated after the stance (ST) phase has been detected at step S140. Here, the variable idxHS may be calculated using the equation “idxHS=wsSW−HS_MARGIN−LECount”, where HS_MARGIN may be set to, for example, “3.” The intention of such a calculation may be easily understood from FIG. 1. The size of the swing period wsSW is continuously counted until the stance (ST) phase is detected after toe off (TO; the start of swing (SW)) has been detected. The corresponding period (between the detection of TO and the detection of ST) includes an interval between heel-strike (HS) and toe ground (TG) (also referred to as ‘HS_MARGIN’) and time LECount required to detect the stance (ST) phase. Therefore, these values HS_MARGIN and LECount are subtracted from wsSW.

In contrast, if heel-strike (HS) has been detected before stance (ST) phase is detected, there is no need to calculate the variable idxHS after the stance (ST) phase has been detected, and thus the preprocessing unit 12 calculates the values of the variables energySumXZ and energySumY using data stored in the buffers bufX[idx], bufY[idx], and bufZ[idx] and the base values energySumBaseXZ and energySumBaseY at step S142.

Thereafter, the control unit 22 analyzes whether the pedestrian walks in a parallel-footed gait pattern (that is, it may be regarded as a healthy gait), using the values of the variables energySumXZ and energySumY calculated by the stance detection unit 16, and displays the results of the analysis to the pedestrian at step S144. Here, the analysis of whether the pedestrian walks in a parallel-footed gait pattern and the display of the analysis results may be sufficiently understood from the above description of the functions of the control unit 22 and the display unit 24 even if an additional description thereof is not given.

Then, the control unit 22 sets the value of the variable LECount containing the count value for the low energy state to “0 (zero)”, and sets the value of a variable HECount containing a count value for a high energy state to “0 (zero)” at step S146.

Thereafter, the high energy state is counted at step S148. The variable HECount is used to perform counting in a high energy state. The purpose of the variable HECount is to automatically detect a case where the axes of the acceleration sensor 10 are seriously changed and then offset values oX, oY, and oZ no longer have meanings. Therefore, it is intended to automatically recalculate offset values and allow offset values to automatically have suitable values under any circumstances.

Thereafter, the control unit 22 is configured to, if the high energy state count value is equal to or greater than a preset maximum threshold MAX_HE_COUNT for high energy count (Yes at step S150), determine that offset recalculation is required at step S512. Here, the preset maximum threshold MAX_HE_COUNT for high energy count may be “150 (corresponding to about 5 seconds because of a sampling rate of 30 Hz)”.

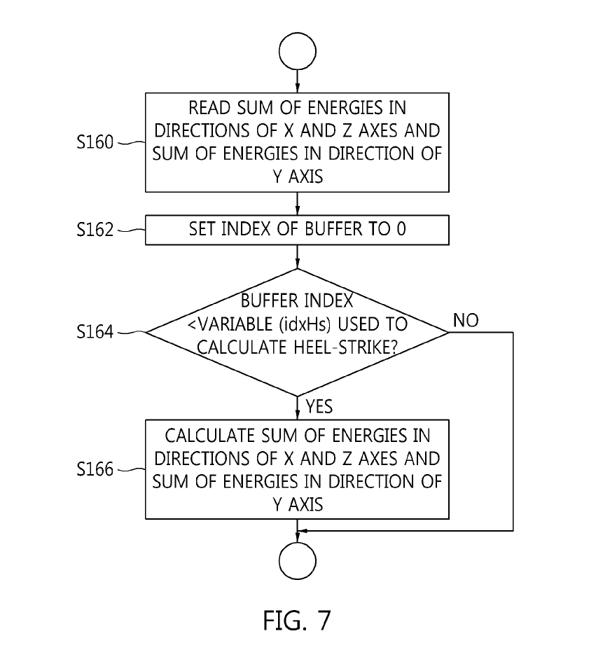

FIG. 7 is a flowchart showing in detail a procedure for calculating the sum of energies in the directions of X and Z axes and the sum of energies in the direction of the Y axis, using the values stored in the buffer of FIG. 6A.

First, it is assumed that a base value energySumBaseXZ is the value of a variable energySumXZ and a base value energySumBaseY is the value of a variable energySumY at step S160.

Thereafter, a buffer index (i) is set to “0 (zero)” at step S162.

Thereafter, the sum of energies in the directions of the X and Z axes and the sum of energies in the direction of the Y axis are calculated using only the data up to the variable idxHS among pieces of buffer data at steps S164 and S166. In this case, the sum of energies in the directions of the X and Z axes is kept in the variable energySumXZ, and the sum of energies in the direction of the Y axis is kept in the variable energySumY. Here, the variable energySumXZ initialized to the base value energySumBaseXZ may be regarded as being obtained while performing additions as in the equation “(bufX[i]−oX)*(bufX[i]−oX)+(bufZ[i]−oZ)*(bufZ[i]−oZ)”, and the variable energySumY initialized to the base value energySumBaseY may be regarded as being obtained while performing additions as in the equation “(bufY[i]−oY)*(bufY[i]−oY)”.

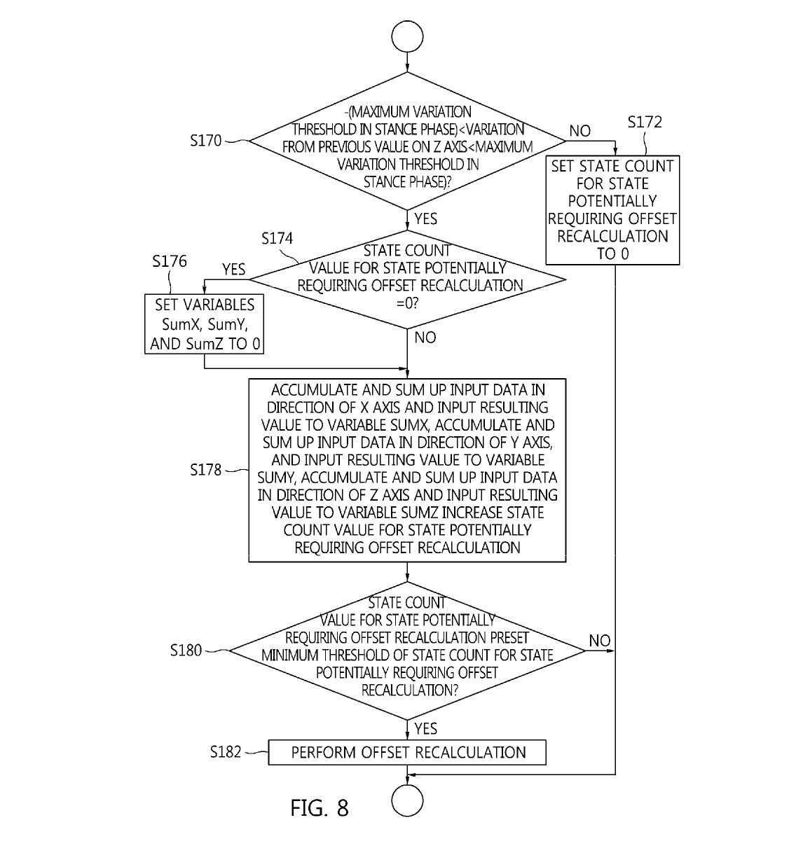

FIG. 8 is a flowchart showing in detail the offset recalculation procedure of FIG. 3. The offset recalculation procedure may be regarded as being processed by the offset recalculation unit 18.

Fist, the offset recalculation unit 18 uses a variation from a previous value on the Z axis so as to determine a state (QST) potentially requiring offset recalculation. That is, the offset recalculation unit 18 compares a variable (gapZ=z−prevZ) having a variation from the previous value on the z axis with a maximum variation threshold MAX_DIFF_ST in the stance (ST) phase. In other words, the offset recalculation unit 18 determines whether the condition “−MAX_DIFF_ST<gapZ<MAX_DIFF_ST” is satisfied at step S170. In this case, the maximum variation threshold MAX_DIFF_ST in the stance (ST) phase denotes the maximum variation of the values of the acceleration sensor 10 in the stance (ST) phase, and may be set to, for example, 50. Of course, instead of the variable gapZ, the value of a variable gapX or a variable gapY may be used. It is important that only one of three variation values having the same effect is used so as to minimize a computational load.

If the condition “−MAX_DIFF_ST<gapZ<MAX_DIFF_ST” is not satisfied, the offset recalculation unit 18 sets a state count QSTCount for the state potentially requiring offset recalculation to “0 (zero)” at step S172.

In contrast, if the condition “−MAX_DIFF_ST<gapZ<MAX_DIFF_ST” is satisfied, the offset recalculation unit 18 determines whether the state count QSTCount for the state potentially requiring offset recalculation is “0 (zero)” at step S174. If the state count QSTCount is 0, each of variables SumX, SumY, and SumZ is set to “0 (zero)” at step S176.

Thereafter, the offset recalculation unit 18 accumulates and sums up input data in the direction of the X axis and sets a resulting value to the value of the variable SumX, accumulates and sums up input data in the direction of the Y axis, and sets a resulting value to the value of the variable SumY, and accumulates and sums up input data in the direction of the Z axis and sets a resulting value to the value of the variable SumZ Further, the state count QSTCount for the state potentially requiring offset recalculation is increased at step S178.

Further, after step S178, the offset recalculation unit 18 determines whether the current value of the state count QSTCount for the state potentially requiring offset recalculation is equal to or greater than a preset minimum threshold MIN_QST_COUNT for the state count QSTCount at step S180. Here, the minimum threshold MIN_QST_COUNT for the state count QSTCount for the state potentially requiring offset recalculation may be preset to “6.”

The embodiment of the present invention determines the state potentially requiring offset recalculation using only the state count QSTCount, thus minimizing the computational load. When the state QST is maintained for a predetermined time MIN_QST_COUNT or longer, offset recalculation is actually performed. Accordingly, if it is determined at step S180 that the value of the state count QSTCount for the state potentially requiring offset recalculation is equal to or greater than the preset minimum threshold MIN_QST_COUNT for the state count potentially requiring offset recalculation, the offset recalculation unit 18 performs offset recalculation at step S182. In this case, an offset (oX) is recalculated as “SumX/QSTCount”, an offset (oY) is recalculated as “SumY/QSTCount”, and an offset (oZ) is recalculated as “SumZ/QSTCount.” Of course, as the offset recalculation is performed, the control unit 22 regards a current gait phase as a stance (ST) phase, and regards a previous gait event as a toe ground (TG) event.

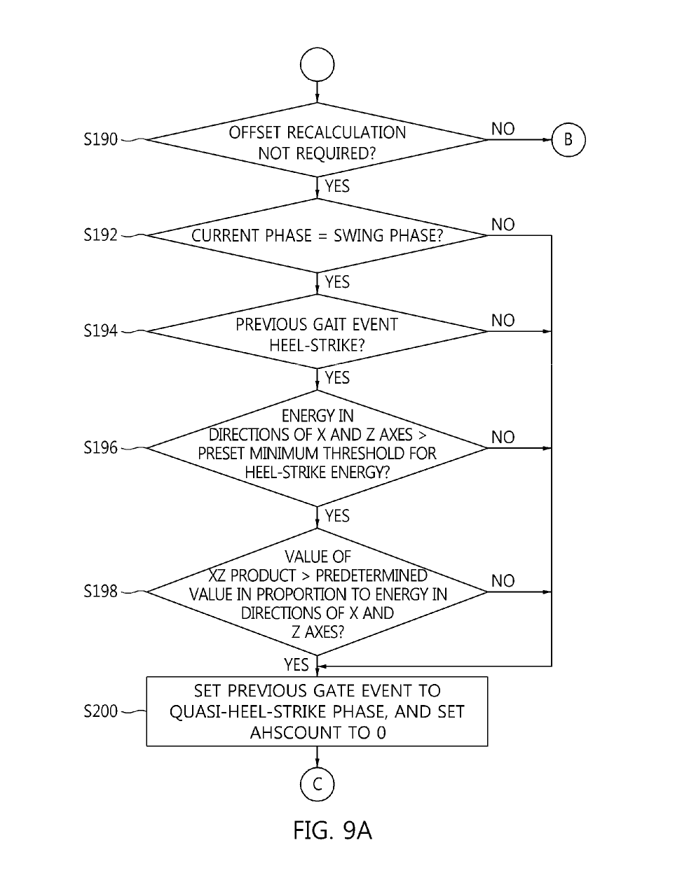

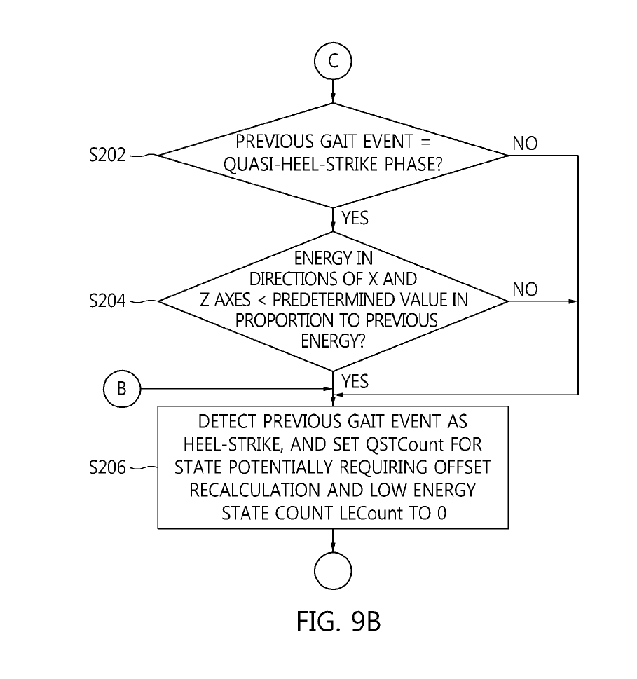

FIGS. 9A and 9B are flowcharts showing in detail the heel-strike detection procedure of FIG. 3. The heel-strike detection procedure may be regarded as being processed by the heel-strike detection unit 20.

First, in order to perform heel-strike (HS) detection, it must be determined whether a current gait phase is a quasi-heel-strike phase QHS.

Accordingly, if offset recalculation is not required (Yes at step S190), and the current gait phase is a swing (SW) phase (Yes at step S192), the heel-strike detection unit 20 may detect heel-strike (HS).

If a previous gait event is not a heel-strike (HS) (Yes at step S194), the heel-strike detection unit 20 determines whether the value of the variable energyXZ indicative of energy on the XZ axes is greater than a preset minimum threshold MIN_HS_ENERGY for heel-strike energy at step S196. The minimum threshold MIN_HS_ENERGY for the heel-strike energy refers to minimum energy in the heel-strike (HS), and may be preset to, for example, “0x100000.”

If the value of the variable energyXZ indicative of energy on the XZ axes is greater than the preset minimum threshold MIN_HS_ENERGY for heel-strike energy, the heel-strike detection unit 20 determines whether an XZ product (that is, the value of the product) based on the acceleration values in the directions of the X and Z axes is greater than a value generated by dividing the value of the variable energyXZ indicative of energy on the XZ axes by a predetermined value R1 at step S198. Here, the predetermined value R1 may be “3.0.”

If the XZ product (that is, the value of the product) based on the acceleration values in the directions of the X and Z axes is greater than the value generated by dividing the value of the variable energyXZ indicative of energy on the XZ axes by the predetermined value R1, the heel-strike detection unit 20 sets a previous gait event to the quasi-heel-strike phase QHS, and sets a count value after heel-strike (AHSCount) to “0 (zero)” at step S200.

In this way, if it is determined that a previous gait event is the quasi-heel-strike phase QHS (Yes at step S202), the heel-strike (HS) may be detected.

Thereafter, if the value of the variable energyXZ indicative of energy on the XZ axes is less than a value generated by dividing a previous value PrevEnergyXZ of the value of the corresponding variable indicative of energy on the XZ axes by a predetermined value R2 (Yes at step S204), the heel-strike detection unit 20 detects the previous gait event of the pedestrian as a heel-strike (HS) phase at step S206. In this case, the predetermined value R2 may be, for example, “3.0.” In this case, it may be understood that the heel-strike detection unit 20 detects heel-strike (HS).

The reason for performing step S204 is to prevent the occurrence of a situation in which heel-strike (HS) is successively detected twice. That is, when a previous event is the heel-strike (HS) phase, the heel-strike (HS) phase is prevented from being detected again.

In this way, after the heel-strike (HS) has been detected, the value of the variable idxHS is calculated. Here, the variable idxHS may be calculated using “idx−AHSCount−HS_MARGIN.” Then, a previous event is set to the heel-strike (HS) phase, the value of the state count QSTCount for the state potentially requiring offset recalculation is set to “0 (zero)”, and the value of the low energy state count LECount is set to “0 (zero)”.

In accordance with the above-described FIGS. 9A and 9B, the heel-strike (HS) procedure may be regarded as being chiefly divided into two stages and performed therein.

First, a quasi-heel-strike phase QHS is detected. That is, in a state in which QHS is not detected, heel-strike (HS) is prevented from being detected. A condition for QHS is given such that the value of the variable energyXZ must be equal to or greater than a predetermined value MIN_HS_ENERGY, and the value of the variable (product) must be equal to or greater than a predetermined value in proportion to the variable energyXZ.

After the detection of QHS, it is recognized that the heel-strike (HS) has been detected only when the value of the variable energyXZ in subsequent input is decreased below a predetermined value in proportion to the value of the previous energy prevEnergyXZ.

In the above flowcharts, although the output of gait analysis information has not been additionally illustrated, it will be sufficiently and easily understood by those skilled in the art from the description of the functions of the control unit 22.

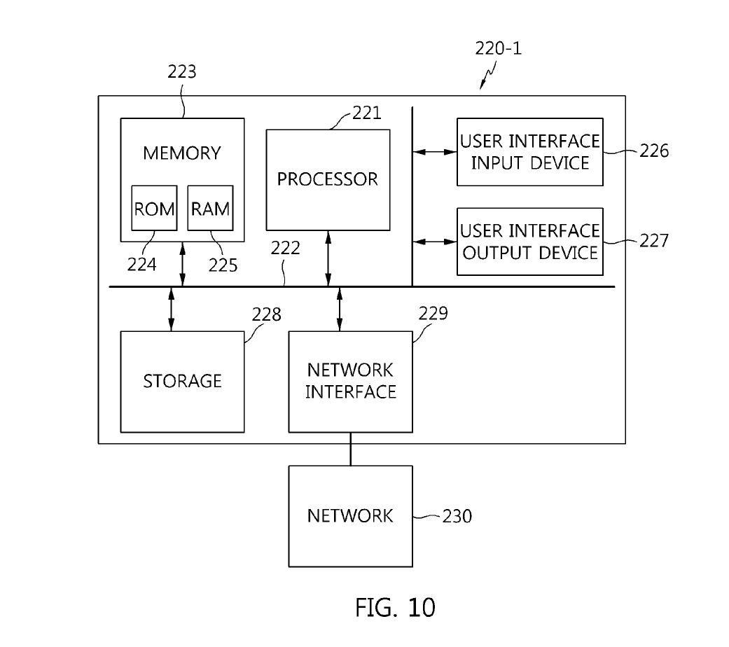

FIG. 10 is an embodiment of the present invention implemented in a computer system.

Referring to FIG. 10, an embodiment of the present invention may be implemented in a computer system, e.g., as a computer readable medium. As shown in FIG. 10, a computer system 220-1 may include one or more of a processor 221, a memory 223, a user input device 226, a user output device 227, and a storage 228, each of which communicates through a bus 222. The computer system 220-1 may also include a network interface 229 that is coupled to a network 230. The processor 221 may be a central processing unit (CPU) or a semiconductor device that executes processing instructions stored in the memory 223 and/or the storage 228. The memory 223 and the storage 228 may include various forms of volatile or non-volatile storage media. For example, the memory may include a read-only memory (ROM) 224 and a random access memory (RAM) 225.

Accordingly, an embodiment of the invention may be implemented as a computer implemented method or as a non-transitory computer readable medium with computer executable instructions stored thereon. In an embodiment, when executed by the processor, the computer readable instructions may perform a method according to at least one aspect of the invention.

In accordance with the present invention having the above configuration, the amount of data to be processed, which is required to detect various types of gait phases and gait events, is minimized, thus enabling a gait to be analyzed at low power and in real time.

Further, by means of the technology of the present invention, the effect of miniaturizing the gait monitoring apparatus may be additionally acquired.

As described above, optimal embodiments of the present invention have been disclosed in the drawings and the specification. Although specific terms have been used in the present specification, these are merely intended to describe the present invention and are not intended to limit the meanings thereof or the scope of the present invention described in the accompanying claims. Therefore, those skilled in the art will appreciate that various modifications and other equivalent embodiments are possible from the embodiments. Therefore, the technical scope of the present invention should be defined by the technical spirit of the claims.

What is claimed is:

1. A gait monitoring apparatus, comprising:
a preprocessing unit for receiving data from a gait detection sensor and preprocessing the data;
a swing detection unit for detecting, based on the data output from the preprocessing unit, whether a current gait phase is a swing phase in which a foot of a pedestrian is lifted from ground and swings in air when the foot of the pedestrian moves forwards;
a stance detection unit for detecting, based on the data output from the preprocessing unit, whether a current gait phase is a stance phase in which the foot of the pedestrian is in contact with the ground;
a heel-strike detection unit for detecting, based on the data output from the preprocessing unit, whether a current gait phase is a heel-strike phase in which the heel of the pedestrian strikes the ground; and
a control unit for determining the current gait phase of the pedestrian, analyzing the gait of the pedestrian, and outputting gait analysis information, based on information output from the preprocessing unit, the swing detection unit, the stance detection unit, and the heel-strike detection unit.

2. The gait monitoring apparatus of claim 1, wherein the preprocessing unit receives sampling data at a sampling rate of 20 to 50 Hz from the gait detection sensor.

3. The gait monitoring apparatus of claim 1, wherein the preprocessing unit receives acceleration data on X, Y, and Z axes from the gait detection sensor, and calculates values of a plurality of variables based on the acceleration data.

4. The gait monitoring apparatus of claim 3, wherein the plurality of variables comprise a variable indicative of energy on XZ axes, a variable indicative of energy on a Y axis, a variable indicative of a product of acceleration data in directions of the X axis and Z axis, and a variable indicative of a variation in the direction of the Z axis.

5. The gait monitoring apparatus of claim 4, wherein the preprocessing unit sets a value, generated by summing up a square of an offset-calibrated value in the direction of the X axis and a square of an offset-calibrated value in the direction of the Z axis, to a value of the variable indicative of energy on the XZ axes.

6. The gait monitoring apparatus of claim 4, wherein the preprocessing unit sets a value, generated by squaring an offset-calibrated value in the direction of the Y axis, to a value of the variable indicative of energy on the Y axis.

7. The gait monitoring apparatus of claim 4, wherein the preprocessing unit sets a value, generated by multiplying an offset-calibrated value in the direction of the X axis by an offset-calibrated value in the direction of the Z axis, to a value of the variable indicative of the product of the acceleration data in the directions of the X and Z axes.

8. The gait monitoring apparatus of claim 4, wherein the swing detection unit is configured to, if a value of the variable indicative of energy on the XZ axes is equal to or greater than a preset minimum energy threshold in a non-stance phase, and if a value of the variable indicative of the product of the acceleration data in the directions of the X and Z axes is a negative value, detect the current gait phase of the pedestrian as the swing phase.

9. The gait monitoring apparatus of claim 8, wherein the control unit determines, based on swing phase detection information output from the swing detection unit, that the current gait phase of the pedestrian is the swing phase, and that a previous gait event is a toe off event.

10. The gait monitoring apparatus of claim 4, wherein the stance detection unit is configured to, if a value of the variable indicative of energy on the XZ axes is less than a preset minimum energy threshold in a non-stance phase, and a count value for a low energy state is equal to or greater than a preset minimum threshold for the low energy count, detect the current gait phase of the pedestrian as the stance phase.

11. The gait monitoring apparatus of claim 10, wherein the control unit determines, based on stance phase detection information output from the stance detection unit, that the current gait phase of the pedestrian is the stance phase, and that a previous gait event is a toe ground event.

12. The gait monitoring apparatus of claim 4, wherein the heel-strike detection unit is configured such that, after it is determined that a value of the variable indicative of energy on the XZ axes is greater than a preset minimum threshold for heel-strike energy and that a value of the variable indicative of the product of the acceleration data in the directions of the X and Z axes is greater than a value generated by dividing the value of the variable indicative of energy on the XZ axes by a predetermined value, if the value of the variable indicative of energy on the XZ axes is less than a value generated by dividing a previous value of the variable indicative of energy on the XZ axes by a predetermined value, a previous gait event of the pedestrian is detected as the heel-strike phase.

13. The gait monitoring apparatus of claim 1, further comprising an offset recalculation unit for recalculating an offset for the data output from the preprocessing unit.

14. The gait monitoring apparatus of claim 13, wherein the offset recalculation unit determines whether a current state is a state potentially requiring offset recalculation, based on one of a variable indicative of a variation in the direction of the X axis, a variable indicative of a variation in the direction of the Y axis, or a variable indicative of a variation in the direction of the Z axis, and performs offset recalculation if the state potentially requiring offset recalculation is maintained for a predetermined period of time or longer, wherein the variables indicative of the variations are output from the preprocessing unit.

15. The gait monitoring apparatus of claim 4, wherein the control unit analyzes whether the pedestrian walks in a parallel-footed gait pattern, based on values of the variable indicative of energy on the XZ axes and the variable indicative of energy on the Y axis, and outputs gait analysis information to a display unit.

16. A gait monitoring method, comprising:
receiving, by a preprocessing unit, data from a gait detection sensor and preprocessing the data;
detecting, by a swing detection unit, whether a current gait phase is a swing phase in which a foot of a pedestrian is lifted from ground and swings in air when the foot of the pedestrian moves forwards, based on the data at preprocessing;
detecting, by a stance detection unit, whether a current gait phase is a stance phase in which the foot of the pedestrian is in contact with the ground, based on the data at preprocessing;
detecting, by a heel-strike detection unit, whether a current gait phase is a heel-strike phase in which the heel of the pedestrian strikes the ground, based on the data at preprocessing; and
determining, by a control unit, the current gait phase of the pedestrian, analyzing the gait of the pedestrian, and outputting gait analysis information, based on information obtained at preprocessing, at detecting the swing phase, at detecting the stance phase, and at detecting the heel-strike phase.

17. The gait monitoring method of claim 16, wherein detecting whether the current gait phase is the swing phase is configured to, if a value of the variable indicative of energy on the XZ axes, generated at preprocessing, is equal to or greater than a preset minimum energy threshold in a non-stance phase, and if a value of the variable indicative of the product of the acceleration data in the directions of the X and Z axes, generated at preprocessing, is a negative value, detect the current gait phase of the pedestrian as the swing phase.

18. The gait monitoring method of claim 16, wherein detecting whether the current gait phase is the stance phase is configured to, if a value of the variable indicative of energy on the XZ axes, generated at preprocessing, is less than a preset minimum energy threshold in a non-stance phase, and a count value for a low energy state is equal to or greater than a preset minimum threshold for the low energy count, detect the current gait phase of the pedestrian as the stance phase.

19. The gait monitoring method of claim 16, wherein detecting whether the current gait phase is the heel-strike phase is configured such that, after it is determined that a value of the variable indicative of energy on the XZ axes, generated at preprocessing, is greater than a preset minimum threshold for heel-strike energy and that a value of the variable indicative of the product of the acceleration data in the directions of the X and Z axes, generated at preprocessing, is greater than a value generated by dividing the value of the variable indicative of energy on the XZ axes by a predetermined value, if the value of the variable indicative of energy on the XZ axes is less than a value generated by dividing a previous value of the variable indicative of energy on the XZ axes by a predetermined value, a previous gait event of the pedestrian is detected as the heel-strike phase.

20. The gait monitoring method of claim 16, wherein outputting the gait analysis information is configured to analyze whether the pedestrian walks in a parallel-footed gait pattern, based on values of the variable indicative of energy on the XZ axes and the variable indicative of energy on the Y axis, the variables being generated at preprocessing.
